(19) United States
(12) Patent Application Publication
(10) Pub. No.: US 2024/0207457 A1
(43) Pub. Date: Jun. 27, 2024

(54) CANCER-SPECIFIC POLYPEPTIDE AND USE THEREOF

(71) Applicant: THE CATHOLIC UNIVERSITY OF KOREA INDUSTRY-ACADEMIC COOPERATION FOUNDATION, Seoul (KR)

(72) Inventors: Jae Myung PARK, Seoul (KR); Moon Hwa KWAK, Seoul (KR)

(21) Appl. No.: 18/288,667

(22) PCT Filed: May 10, 2022
(86) PCT No.: PCT/KR2022/006693

§ 371 Date: Oct. 27, 2023

(30) Foreign Application Priority Data
May 11, 2021 (KR) 10-2021-0060992
Apr. 14, 2022 (KR) 10-2022-0046578

(51) Int. Cl. A61K 51/08; A61K 41/00; A61K 49/00; A61P 35/00

(52) U.S. Cl. CPC A61K 51/08; A61K 41/0061; A61K 41/0071; A61K 41/0076; A61K 49/0056; A61P 35/00

(57) ABSTRACT

An aspect relates to a cancer-specific polypeptide consisting of an amino acid sequence of SEQ ID NO: 1. The polypeptide according to an aspect may bind specifically to LGR5 protein expressed in a tumor tissue, and when binding to a labeling material (for example, a fluorescent material), a cancer that expresses the LGR5 protein may be diagnosed. Furthermore, when the polypeptide binds to an isotope, a metastatic tumor may be also diagnosed. When the polypeptide according to an aspect binds to a photosensitizer and is then administered to a subject, the photosensitizer may be activated through light irradiation so that cancer cells may be killed, thereby enabling the prevention, amelioration, or treatment of cancer.

### TECHNICAL FIELD

Proposed are a cancer-specific polypeptide and a use thereof to diagnose, prevent, or treat cancer.

### BACKGROUND ART

Cells, the smallest unit constituting the human body, normally maintain cell number balance while dividing, growing, and dying through intracellular regulatory functions. However, cancer is defined as a condition in which abnormal cells whose proliferation and inhibition are not controlled due to various reasons not only proliferate excessively but also invade surrounding tissues and organs, resulting in tumefaction and destruction of normal tissues. Cancer means the uncontrolled proliferation of cells as described above and destroys the structure and function of normal cells and organs, so the diagnosis and treatment thereof are significantly important.

On the other hand, therapeutic responsiveness varies due to genetic differences among individual cancer patients, so treatment cases differ for each patient, causing problems in treatment. Hence, for effective cancer patient treatment, there is a demand for developing functional targeted therapeutic agents capable of targeting cancer microenvironments that vary with radiation responsiveness and biomarkers depending on the environment. Through this, diagnosis and treatment can be customized for each patient.

In addition, drug delivery systems or targeted therapies that selectively deliver drugs to cancer cells and tissues are technologies that have received a great deal of attention. This is because even when using the same amount of anticancer drug, drug potency can be improved while significantly reducing the side effects on normal tissues. Furthermore, when applied to gene therapy, viruses may be selectively delivered to cancer cells, thereby improving the treatment efficiency while reducing serious side effects. To this end, antigens specific to tumor cells and antibodies targeting the same have been mainly developed until now. However, in the case of antibodies, there are problems such as the risk of immune response and low efficiency of infiltration into tissues. On the other hand, peptides have the following advantages: small molecular weights, less risk of immune response, and easy penetration into tissues. Therefore, a cancer-targeting peptide linked to an existing anticancer drug can be used as an intelligent drug delivery system for selectively delivering drugs to tumors (Polymer Science and Technology, Volume 25, Number 4, August 2014, 283 p to 289 p).

### DISCLOSURE

### Technical Problem

One aspect aims to provide a polypeptide having an amino acid sequence of SEQ ID NO: 1.

Another aspect aims to provide a composition for diagnosing cancer, the composition containing a polypeptide having an amino acid sequence of SEQ ID NO: 1.

A further aspect provides a method of diagnosing cancer, the method including: administering a polypeptide having an amino acid sequence of SEQ ID NO: 1 to a subject; and confirming a position of the polypeptide in the subject.

A further aspect aims to provide a pharmaceutical composition for preventing or treating cancer, the composition containing a polypeptide having an amino acid sequence of SEQ ID NO: 1.

A further aspect provides a method of preventing or treating cancer, the method including: administering a polypeptide having an amino acid sequence of SEQ ID NO: 1 to a subject in need thereof; and irradiating the subject with light.

A further aspect aims to provide a use of a polypeptide having an amino acid sequence of SEQ ID NO: 1 to prepare a drug for preventing or treating cancer.

A further aspect aims to provide a health functional food for preventing or improving cancer, the food containing a polypeptide having an amino acid sequence of SEQ ID NO: 1.

### Technical Solution

One aspect provides a polypeptide having an amino acid sequence of SEQ ID NO: 1.

The term “amino acid”, the basic structural unit of protein, means an organic acid that contains both amino and carboxyl groups.

The term “polypeptide” means a straight-chain molecule formed by linking amino acid residues with each other by peptide bonds and may be used interchangeably with a “peptide”.

The polypeptide may include polypeptides each independently having a sequence homology of about 70% or more, about 75% or more, about 80% or more, about 85% or more, about 90% or more, about 92% or more, about 95% or more, about 97% or more, about 98% or more, and about 99% or more to the amino acid sequence of SEQ ID NO: 1.

In addition, in one aspect, the polypeptide may include the polypeptide having the amino acid sequence of SEQ ID NO: 1 and a functional variant thereof. The functional variant means all similar sequences in which some amino acid substitutions occur at amino acid positions without affecting the biological nature of the polypeptide according to one aspect.

The term “homology” is intended to show the similarity to the wild-type amino acid sequence. Comparison of such homology may be performed using a comparison program widely known in the art, and homology between two or more sequences may be calculated in percentage (%).

Furthermore, to obtain better chemical stability, enhanced pharmacological properties (such as half-life, absorption, titer, potency, and the like), altered specificity (for example, broad biological activity spectrum), and reduced antigenicity, a protecting group may bind to the N- or C-terminus. The protecting group may be an acetyl group, fluorenyl methoxy carbonyl group, formyl group, palmitoyl group, myristyl group, stearyl group, or polyethylene glycol (PEG). However, any component capable of improving the modification of the polypeptide, especially the stability of the polypeptide, may be included without limitation.

The term “stability” may not only mean in vivo stability, which protects the polypeptide from attack by in vivo proteases, but also mean storage stability (for example, storage stability at room temperature).

In one aspect, the polypeptide may be derived from nature or obtained by various well-known methods of synthesizing polypeptides in the art. As one example, the polypeptide may be prepared using genetic recombination and protein expression systems. Alternatively, the polypeptide may be prepared by in vitro synthesis methods through chemical synthesis, such as protein synthesis (for example, liquid- or solid-phase synthesis, fragment condensation, and F-MOC or T-BOC chemistry), and cell-free protein synthesis methods. In addition, as one example, the polypeptide may be a peptide, an extract of plant-derived tissues or cells, or a product obtained by culturing microorganisms (for example, bacteria or fungi, and especially yeast).

The polypeptide may effectively target cancer cells by specifically binding to leucine-rich repeat-containing G-protein coupled receptor 5 (LGR5) protein expressed by the cancer cells and thus may be effectively used for diagnosing, preventing, or treating cancer.

The polypeptides may be selected by phage display. Specifically, the polypeptide may be selected by repeatedly performing a process of selecting only phages attached to the LGR5 protein among a phage library and selecting phages having a high attachment ability.

According to one example, as a result of selecting phages having a higher absorbance at a wavelength of 450 nm in the LGR5 protein than in the BSA protein by phage display, it was confirmed that the polypeptide having the amino acid sequence of SEQ ID NO: 1 had a high attachment ability to the LGR5 protein (see Example 1).

According to one example, as a result of binding FITC, a fluorescent material, to the polypeptide, treating gastric cancer cell lines and a normal cell line with the resulting polypeptide, and then performing immunochemical staining, the fluorescence intensity was measured to be high in the gastric cancer cell lines containing LGR5 compared to that in the normal cell line free of LGR5, confirming that the polypeptide had a high attachment ability to the LGR5 protein (see Example 2).

Another aspect provides a composition for diagnosing cancer, the composition containing a polypeptide having an amino acid sequence of SEQ ID NO: 1.

The “amino acid”, “polypeptide”, and the like may fall within the scopes described above.

In addition to the polypeptide, the composition may contain a buffer solution (for example, phosphate, citrate, acetate, or the like) that allows the structure or physiological activity of the peptide to be stably maintained

In one aspect, the composition may further contain a fluorescent material. Specifically, the fluorescent material may bind to the polypeptide through bonding, and the bonding may include various bonds, such as a hydrogen bond, a covalent bond, an ionic bond, an electrostatic attraction, and the like.

In addition, in one aspect, the fluorescent material may include one or more selected from the group consisting of xanthene derivatives, cyanine derivatives, oxadiazole derivatives, acridine derivatives, arylmethine derivatives, tetrapyrrole derivatives, near-infrared fluorophores (NIR fluorophores), chlorin e6 (Ce6), and green fluorescent protein (GFP).

Specifically, the xanthine derivatives may include one or more selected from the group consisting of fluorescein, Oregon Green, and Texas Red;
the cyanine derivatives may include one or more selected from the group consisting of cyanine 2 (Cy2), Cy3, Cy3B, Cy3.5, Cy5, Cy5.5, Cy7, indocarbocyanine, rhodamine, oxacarbocyanine, thiacarbocyanine, and merocyanine;
the oxadiazole derivatives may include one or more selected from the group consisting of pyrodyloxazole, nitrobenzoxadiazole, and benzoxadiazole;
the acridine derivatives may include one or more selected from the group consisting of Nile red, Nile orange, and acridine yellow;
the arylmethine derivatives may include one or more selected from the group consisting of aumarine, crystal violet, and malachite green;
the tetrapyrrole derivative may include one or more selected from the group consisting of porphin, phthalocyanine, and bilirubin; and
the NIR fluorophores may include one or more selected from the group consisting of X-SIGHT, Pz 247, DyLight 750, DyLight 800, Alexa Fluor 680, Alexa Fluor 750, IRDye 680, IRDye 800CW, indocyanine green, and zwitterionic near-infrared fluorophores. More specifically, the fluorescent material may include one or more selected from the group consisting of FITC, Cy5.5, Ce6, and GFP.

The term “cancer” means a condition in which abnormal cells that are required to be killed proliferate excessively and invade surrounding tissues and organs, thereby destroying or transforming existing structures, which includes all tumors, neoplasms, benign tumors, malignant tumors, carcinoma, sarcoma, and the like. In addition, the term “cancer” is used interchangeably with a “tumor”.

In one aspect, the cancer may express leucine-rich repeat-containing G-protein coupled receptor 5 (LGR5) protein. The “LGR5 protein”, one type of GPCR class A receptor protein, may be encoded by the LGR5 gene and is generally understood as a biomarker of adult stem cells.

In one aspect, the polypeptide may bind to the LGR5 protein of the cancer through bonding, and the bonding may include various bonds, such as a hydrogen bond, a covalent bond, an ionic bond, an electrostatic attraction, and the like.

According to one aspect, the cancer may include one or more selected from the group consisting of gastric cancer, colon cancer, pancreatic cancer, liver cancer, cervical cancer, breast cancer, ovarian cancer, head and neck cancer, carcinoid, prostate cancer, lung cancer, bladder cancer, endometrial cancer, melanoma, kidney cancer, testicular cancer, glioma, thyroid cancer, skin cancer, and lymphoma. Specifically, the cancer may include one or more selected from the group consisting of gastric cancer, colon cancer, pancreatic cancer, liver cancer, cervical cancer, breast cancer, ovarian cancer, head and neck cancer, carcinoid, prostate cancer, lung cancer, bladder cancer, endometrial cancer, melanoma, kidney cancer, and testicular cancer. More specifically, the cancer may include one or more selected from the group consisting of gastric cancer, colon cancer, pancreatic cancer, liver cancer, cervical cancer, breast cancer, ovarian cancer, head and neck cancer, carcinoid, and prostate cancer.

In addition, in one aspect, the cancer may be a metastatic tumor, and specifically, the metastatic tumor may be an intraperitoneal metastatic tumor. The term “metastatic tumor” means a new tumor formed when primary tumor cells flow through the blood flow or lymph flow and are then gradually infiltrated, transmitted, and settled into surrounding tissues.

The term “diagnosing” means to determine whether a subject currently has a specific disease or condition as well as to determine the prognosis of a subject suffering from a specific disease or condition.

The polypeptide may specifically bind to the leucine-rich repeat-containing G-protein coupled receptor 5 (LGR5) protein expressed by cancer cells and thus may effectively target the cancer cells. When the composition contains the fluorescent material, fluorescence may be expressed in the targeted cancer cell tissue. In addition, all cancers, including localized and metastatic tumors, expressing the LGR5 protein may be diagnosable by confirming the expressed fluorescence intensity.

According to one example, as a result of binding FITC to the polypeptide and then treating a gastric cancer cell line and a normal cell line with the resulting peptide by concentrations, the fluorescence intensity was measured to be high in the gastric cancer cell lines containing LGR5 compared to that in the normal cell lines free of LGR5, confirming that the polypeptide was capable of being attached to the LGR5 protein in a concentration-dependent manner (see Example 3).

According to another example, as a result of binding Cy5.5, a fluorescent material, to the polypeptide and administering the resulting polypeptide to mice with a xenograft of a gastric cancer cell line, the fluorescence intensity was measured to be significantly high in the tumor tissue compared to that in other tissues, confirming that the polypeptide was capable of diagnosing the tumor tissue in vivo (see Example 4).

According to another example, as a result of binding Ce6, a fluorescent material, to the polypeptide and administering the resulting polypeptide to mice with a xenograft of a gastric cancer cell line, the fluorescence intensity was measured to be significantly high in the tumor tissue compared to that in other tissues, confirming that the polypeptide was capable of diagnosing the tumor tissue in vivo. In addition, as a result of extracting organs from the subject and also analyzing the fluorescence intensity ex vivo, it was confirmed that the fluorescence intensity was measured to be significantly high in the tumor tissue compared to that in other tissues (see Example 6).

According to a further example, as a result of binding FITC, the fluorescent material, to the polypeptide and treating tumor tissue with either the resulting polypeptide or a negative control peptide-FITC composite, even though no fluorescence was observed in the case of the group treated with the negative control, the fluorescence intensity appeared to be significantly high in the tumor tissue in the case of the group treated with the polypeptide of the present disclosure, confirming that the polypeptide was capable of targeting the tumor tissue (see Example 8).

According to one example, as a result of binding Chlorin e6 (Ce6) to the polypeptide and administering the resulting polypeptide to an intraperitoneal metastasis mouse model with a gastric cancer cell line, the concentration of Ce6 in the tumor was measured to be high compared to that in the case of the group treated with Ce6 alone, confirming that the polypeptide was also capable of diagnosing the metastatic tumor (see Example 9).

In addition, in one aspect, the composition may bind to an isotope. Specifically, the isotope may include one or more selected from the group consisting of ¹¹C, ¹³N, ¹⁸F, ⁶⁸Ga, ⁶¹Cu, ¹²⁴I, ¹²⁵I, ¹¹¹In, ^{99m}Tc, ³²P, and ³⁵S and more specifically be ¹¹¹In.

According to one example, as a result of binding ¹¹¹In, an isotope, to the polypeptide and administering the resulting polypeptide to the intraperitoneal metastasis mouse model, the fluorescence intensity was measured to be higher in the abdominal cavity than in the case of normal animals, confirming that the polypeptide was also capable of diagnosing the metastatic tumor (see Example 10).

A further aspect provides a method of diagnosing cancer, the method including: administering a polypeptide having an amino acid sequence of SEQ ID NO: 1 to a subject; and confirming a position of the polypeptide in the subject.

The “amino acid”, “polypeptide”, “cancer”, “diagnosing”, and the like may fall within the scopes described above.

The term “subject” means a subject in need of cancer treatment. Specifically, the subject means humans or mammals such as primates, mice, rats, dogs, cats, horses, pigs, rabbits, cows, and the like.

The term “administering” means to physically introduce a pharmaceutical composition into a subject using any of the various methods and delivery systems known to those skilled in the art.

In one aspect, the confirming of the position may be performed by measuring a signal expressed by the polypeptide or a signal expressed by a labeling material after the polypeptide binds to the labeling material, using the nature of the polypeptide to specifically bind to the LGR5 protein, expressed by cancer cells. The labeling material may contain a fluorescent material and an isotope. When the signal expressed by the polypeptide or labeling material in a specific tissue is significantly larger than that in other tissues, the corresponding tissue may be diagnosed as tumor tissue.

A further aspect provides a pharmaceutical composition for preventing or treating cancer, the composition containing a polypeptide having an amino acid sequence of SEQ ID NO: 1.

The “amino acid”, “polypeptide”, “cancer”, and the like may fall within the scopes described above.

The term “preventing” means all actions that suppress or delay the onset of cancer, and the term “treating” means all actions that cure or beneficially change cancer by administering the pharmaceutical composition according to one aspect.

In one aspect, the cancer may include one or more selected from the group consisting of gastric cancer, colon cancer, pancreatic cancer, liver cancer, cervical cancer, breast cancer, ovarian cancer, head and neck cancer, carcinoid, prostate cancer, lung cancer, bladder cancer, endometrial cancer, melanoma, kidney cancer, testicular cancer, glioma, thyroid cancer, skin cancer, and lymphoma. Specifically, the cancer may include one or more selected from the group consisting of gastric cancer, colon cancer, pancreatic cancer, liver cancer, cervical cancer, breast cancer, ovarian cancer, head and neck cancer, carcinoid, prostate cancer, lung cancer, bladder cancer, endometrial cancer, melanoma, kidney cancer, and testicular cancer. More specifically, the cancer may include one or more selected from the group consisting of gastric cancer, colon cancer, pancreatic cancer, liver cancer, cervical cancer, breast cancer, ovarian cancer, head and neck cancer, carcinoid, and prostate cancer.

In one aspect, the pharmaceutical composition may further contain a photosensitizer. The term “photosensitizer” means a chemical compound that generates oxygen and other materials capable of killing cells or microorganisms by absorbing the energy of light having a specific wavelength and delivering the energy to other molecules.

In one aspect, the photosensitizer may bind to the polypeptide through bonding, and the bonding may include various bonds, such as a hydrogen bond, a covalent bond, an ionic bond, an electrostatic attraction, and the like.

In addition, in one aspect, the photosensitizer may include one or more selected from the group consisting of hematoporphyrin and derivatives thereof, 5-aminolevulinic acid and derivatives thereof, chlorin and derivatives thereof, purpurin and derivatives thereof, benzoporphyrin and derivatives thereof, phthalocyanine and derivatives thereof, and texaphyrin and derivatives thereof. The photosensitizer may be specifically chlorin or derivatives thereof and more specifically chlorin e6 (Ce6).

In one aspect, the photosensitizer may be activated by light to kill cancer cells. The term “light” means all electromagnetic waves, including X-rays, ultraviolet rays, visible rays, and ultraviolet rays, and light irradiation may be performed using a laser device. The term “activated” means a state where the photosensitizer absorbs light energy of a specific wavelength to kill cells or microorganisms.

The polypeptide may specifically bind to leucine-rich repeat-containing G-protein coupled receptor 5 (LGR5) protein expressed by cancer cells and thus may effectively target the cancer cells. When the pharmaceutical composition contains the photosensitizer, the photosensitizer is activated with light irradiation, and the targeted cancer cell tissue is effectively killed, thereby exhibiting cancer prevention or treatment effects.

According to one example, as a result of binding Ce6, a photosensitizer, to the peptide, treating a gastric cancer cell line with the resulting polypeptide, and then irradiating the cell line with a laser beam, the cells were killed, unlike in the case of the group treated with Ce6 alone, and reactive oxygen species (ROS) were detected, confirming that the polypeptide was capable of killing the cancer cells (see Example 5).

According to another example, as a result of binding Ce6 to the polypeptide, administering the resulting polypeptide to mice with a xenograft of a gastric cancer cell line, and then irradiating the mice with a laser beam, the size of the tumor was significantly reduced compared to that in the case of the control group, confirming that the polypeptide was capable of treating the localized tumor in vivo (see Example 7).

The “pharmaceutical composition” may contain an active ingredient alone or may be provided as a pharmaceutical composition containing one or more pharmaceutically acceptable carriers, excipients, or diluents.

Specifically, the carrier may, for example, have a form of a colloidal suspension, powder, saline solution, lipid, liposome, microsphere, or nano-spherical particle. The carrier may form a complex with a carrier means or be related thereto. In addition, the carrier may be delivered in vivo using well-known delivery systems in the art, such as lipids, liposomes, microparticles, gold, nanoparticles, polymers, condensation agents, polysaccharides, polyamino acids, dendrimers, saponins, adsorption enhancers, or fatty acids.

When formulating the pharmaceutical composition, commonly used diluents or excipients, such as lubricants, sweeteners, flavoring agents, emulsifiers, suspending agents, preservatives, fillers, extenders, binders, wetting agents, disintegrating agents, and surfactants, may be used for preparations. Examples of solid preparations for oral administration may include tablets, pills, powders, granules, capsules, and the like. In addition, such solid preparations may be prepared by mixing one or more excipients, such as starch, calcium carbonate, sucrose or lactose, gelatin, and the like, in the pharmaceutical composition. Furthermore, in addition to simple excipients, lubricants, such as magnesium stearate and talc, are also usable. Examples of liquid preparations for oral use include suspensions, liquid preparations for internal use, emulsions, syrups, and the like. In addition to commonly used diluents, such as water and liquid paraffin, various excipients, for example, wetting agents, sweeteners, aromatics, preservatives, and the like, may be included. Examples of preparations for parenteral administration may include sterile aqueous solutions, non-aqueous solvents, suspensions, emulsions, freeze-dried preparations, and suppositories. As for non-aqueous solvents and suspending agents, propylene glycol, polyethylene glycol, vegetable oils such as olive oil, injectable esters such as ethyl oleate, and the like may be used. As for suppository bases, Witepsol, macrogol, Tween 61, cacao fat, laurin fat, glycerogeratin, and the like may be used. In addition, when being prepared in the form of a collyrium, known diluents or excipients may be used.

The pharmaceutical composition may be administered parenterally by external skin application or an injection method, such as intraperitoneal injection, rectal injection, subcutaneous injection, intravenous injection, intramuscular injection, intra-arterial injection, intramedullary injection, intracardiac injection, intrathecal injection, transdermal injection, intranasal injection, enteral injection, topical injection, sublingual injection, or intrathoracic injection. In addition, the pharmaceutical composition may be administered through oral, rectal, inhalation, or internasal administration.

The pharmaceutical composition is administered in a pharmaceutically effective amount. The term “pharmaceutically effective dose” means an amount sufficient to treat or diagnose a disease at a reasonable benefit-risk ratio applicable to medical treatment. The effective dose level may be determined according to types of disease, severity, drug activity, sensitivity to a drug, administration time, administration route and rate of release, duration of treatment, factors including concurrent medications, and other factors well-known in the medical field. The administration may be performed once a day or in several divided doses. For example, the administration may be performed every other day or once a week.

In one aspect, the polypeptide binding to the photosensitizer may be administered in an amount of 0.1 to 10 mg/kg, specifically, 0.1 to 10 mg/kg, 0.1 to 9 mg/kg, 0.1 to 7 mg/kg, 1 to 10 mg/kg, 1 to 9 mg/kg, 1 to 7 mg/kg, 3 to 10 mg/kg, 3 to 9 mg/kg, or 3 to 7 mg/kg.

When administering the polypeptide binding to the photosensitizer in an amount of less than 0.1 mg/kg, the effective amount of the photosensitizer may be insufficient, so the effect of killing cancer cells may be reduced. When administering the polypeptide binding to the photosensitizer in an amount exceeding 10 mg/kg, excessive phototoxicity of the photosensitizer may result in cytotoxicity or cause the photosensitizer to be excessively accumulated in the body.

The pharmaceutical composition may be provided by being mixed with a known pharmaceutical composition for preventing or treating cancer in the related art or a newly developed pharmaceutical composition for preventing or improving cancer. When the pharmaceutical composition further contains a pharmaceutical composition for preventing or improving cancer, it is important to mix the pharmaceutical composition in the minimum amount that may enable the maximum effect to be obtained without causing side effects, which may be easily determined by those skilled in the art.

The pharmaceutical composition may be administered alone or in combination with other anticancer drugs. In other words, the pharmaceutical composition may be administered in combination with other anticancer drugs or known compositions having cancer prevention or treatment effects, administered simultaneously, separately, or sequentially, and administered once or multiple times. Considering all the factors described above, it is important to administer the composition in the minimum amount that may enable the maximum effect to be obtained without causing side effects, which may be easily determined by those skilled in the art.

A further aspect provides a method of preventing or treating cancer, the method including: administering a polypeptide having an amino acid sequence of SEQ ID NO: 1 to a subject in need thereof; and irradiating the subject with light.

The “amino acid”, “polypeptide”, “subject”, “diagnosing”, “administrating”, “light”, “cancer”, “preventing”, “treating”, and the like may fall within the scopes described above.

The irradiating of the subject with the light may be performed using a laser device and may be directly performed on a specific part of the subject or on the entire body of the subject, which serves to deliver the light energy to the cancer cells binding to the polypeptide.

In one aspect, when the photosensitizer binds to the polypeptide, the photosensitizer is activated with light irradiation, and the targeted cancer cell tissue is effectively killed, thereby exhibiting cancer prevention or treatment effects.

A further aspect provides a use of a polypeptide having an amino acid sequence of SEQ ID NO: 1 to prepare a drug for preventing or treating cancer.

The “cancer”, “preventing”, “treating”, “amino acid”, “polypeptide”, and the like may fall within the scopes described above.

In one aspect, when the photosensitizer binds to the polypeptide, the photosensitizer is activated with light irradiation, and the targeted cancer cell tissue is effectively killed, thereby exhibiting cancer prevention or treatment effects.

A further aspect provides a health functional food for preventing or improving cancer, the food containing a polypeptide having an amino acid sequence of SEQ ID NO: 1.

The “amino acid”, “polypeptide”, “cancer”, “preventing”, and the like may fall within the scopes described above.

The term “improving” may mean all actions that result in at least a reduction in the severity of parameters related to the condition being treated, for example, symptoms. In this case, for cancer prevention or improvement, the health functional food may be used simultaneously or separately with drugs for treatment before or after the onset of the corresponding disease.

In the health functional foods described above, an active ingredient may be added directly to the food or used in combination with other foods or food ingredients and may be used appropriately according to existing methods. The amount of the active ingredient mixed may be appropriately determined depending on the purpose of use (prevention or improvement). Generally, when manufacturing food or beverages, the health functional food may be added in an amount of about 15% by weight or less, more specifically, about 10% by weight or less, with respect to the weight of raw materials. However, in the case of long-term intake for the purpose of health and hygiene or the purpose of health control, the amount may be lower than the range described above.

The health functional food may be formulated as one type selected from the group consisting of tablets, pills, powders, granules, powders, capsules, and liquid formulations by further containing one or more among carriers, diluents, excipients, and additives. Examples of foods to which compounds, according to one aspect, may be added include various foods, powders, granules, tablets, capsules, syrups, beverages, gum, tea, vitamin complexes, health functional foods, and the like.

Specific examples of the carriers, excipients, diluents, and additives may include at least one selected from the group consisting of lactose, dextrose, sucrose, sorbitol, mannitol, erythritol, starch, gum acacia, calcium phosphate, alginate, gelatin, calcium silicate, microcrystalline cellulose, polyvinylpyrrolidone, cellulose, water, sugar syrup, methylcellulose, methyl hydroxybenzoate, propyl hydroxybenzoate, talc, magnesium stearate, and mineral oil.

The health functional food may contain other ingredients, in addition to the active ingredient described above, as active ingredients without any particular limitation. For example, like regular beverages, various flavoring agents or natural carbohydrates may be contained as additional ingredients. Examples of the natural carbohydrates described above may include monosaccharides, for example, fructose, and the like; disaccharides, for example, maltose, sucrose, and the like; and polysaccharides, for example, common sugars, such as dextrins and cyclodextrins, and sugar alcohols, such as xylitol, sorbitol, and erythritol. As flavoring agents other than those described above, natural flavoring agents (thaumatin, stevia extract (for example, rebaudioside A, glycyrrhizin, and the like)) and synthetic flavoring agents (saccharin, aspartame, and the like) may be beneficially used. The ratio of the natural carbohydrates may be appropriately determined by the selection of those skilled in the art.

In addition to those described above, the health functional food, according to one aspect, may contain various nutrients, vitamins, minerals (electrolytes), flavoring agents, such as synthetic and natural flavors, colorants, thickeners (cheese, chocolate, and the like), pectic acid and salts thereof, alginic acid and salts thereof, organic acids, protective colloidal thickeners, pH modifiers, stabilizers, preservatives, glycerin, alcohol, carbonating agents used in carbonated beverages, and the like. Such components may be used independently or in combination, and the ratio of such additives may also be appropriately selected by those skilled in the art.

The health functional food may be provided in combination with a known health functional food for preventing or improving cancer in the related art or a newly developed health functional food for preventing or improving cancer. When the health functional food further contains a health functional food for preventing or improving cancer, it is important to mix the health functional food in the minimum amount that may enable the maximum effect to be obtained without causing side effects, which may be easily determined by those skilled in the art.

In addition, the health functional food may be consumed alone or in combination with the health functional food for preventing or improving cancer. The health functional food may be consumed in combination with known compositions having the effect of preventing or improving cancer or other health functional foods for preventing or improving cancer, consumed simultaneously, separately, or sequentially, and consumed once or multiple times. Considering all the factors described above, it is important to consume the health functional food in the minimum amount that may enable the maximum effect to be obtained without causing side effects, which may be easily determined by those skilled in the art.

### Advantageous Effects

A polypeptide, according to one aspect, may specifically bind to the LGR5 protein expressed by tumor tissues and may bind to a labeling material (for example, a fluorescent material), thereby enabling LGR5 protein-expressing cancers to be diagnosable. Furthermore, the polypeptide may bind to an isotope, thereby enabling metastatic tumors to be diagnosable. Moreover, when binding the polypeptide, according to one aspect, to a photosensitizer and administering the resulting polypeptide to a subject, the photosensitizer is activated with light irradiation to kill cancer cells, thereby enabling cancer prevention, improvement, or treatment.

### DESCRIPTION OF DRAWINGS

FIG. 1 shows target phages and a peptide having a high attachment ability to LGR5, a gastric cancer cell marker, selected using phage display;

FIG. 2 shows results of immunochemical staining performed by binding FITC to a target phage and a peptide having a high attachment ability to LGR5;

FIG. 3 shows results of immunochemical staining performed on FITC-peptide in HEK293T kidney cells, not in gastric cancer cell lines;

FIG. 4 shows results of measuring the degree of saturation in fluorescence density of FITC-peptide in a gastric cancer cell line and a normal cell line;

FIGS. 5A and 5B show results of measuring fluorescence density values of FITC-peptide in a gastric cancer cell line and a normal cell line through a flow cytometer;

FIGS. 6A and 6B show results of measuring fluorescence density values of a group administered a control peptide and a group administered a developed peptide in mouse organs implanted with a gastric cancer cell line;

FIG. 7 shows results of measuring fluorescence density values of Ce6-peptide in gastric cancer cell lines and a normal cell line;

FIGS. 8A and 8B show cytotoxicity analysis results with or without involving laser irradiation after treating a gastric cancer cell line with Ce6 and Ce6-peptide by concentrations;

FIG. 9 shows ROS detection results confirmed by treating a gastric cancer cell line with Ce6-peptide, irradiating the resulting cell lines with a laser beam, and then performing DCF-DA staining;

FIGS. 10A and 10B show results of measuring fluorescence values of a group administered Ce6 alone and a group administered Ce6-peptide in mouse tumors implanted with a gastric cancer cell line;

FIGS. 11A and 11B show results of measuring fluorescence values of a group administered Ce6 alone and a group administered Ce6-peptide by administering Ce6 and Ce6-peptide to mice implanted with a gastric cancer cell line and then extracting organs;

FIGS. 12A and 12B show results of tumor sizes depending on each treatment, measured by dividing mice implanted with a gastric cancer cell line into four groups: an untreated group, a group irradiated with a laser beam, a group irradiated with a laser beam after Ce6 administration, and a group irradiated with a laser beam after Ce6-peptide administration;

FIG. 13 shows results of measuring fluorescence values after treating a synthesized tumor with control peptide-FITC and target peptide-FITC;

FIGS. 14A and 14B show an intraperitoneal metastasis model confirmed by injecting a GFP-labeled gastric cancer cell line into mice;

FIGS. 15A and 15B show results of measuring tumor-targeting ability after each independently treating modeled mice with intraperitoneal metastasis with Ce6 and Ce6-peptide;

FIG. 16 shows results of measuring fluorescence values after injecting isotope ¹¹¹In-peptide into an intraperitoneal metastasis model with a gastric cancer cell line; and

FIG. 17 shows results of ¹¹¹In-peptide distribution in tissue confirmed 24 hours after injecting isotope ¹¹¹In-peptide into an intraperitoneal metastasis model with a gastric cancer cell line.

### MODE FOR INVENTION

Hereinafter, the present disclosure will be described in more detail through examples. However, the following examples are disclosed for illustrative purposes, and the scope of the present disclosure is not limited by the following examples.

### Example

### 1. Selection of Gastric Cancer Cell Line-Targeting Phages

Selection by phage display was performed to select phages targeting a gastric cancer cell line. A phage library (Ph.D.TM-7 phage library, Cat No. E8100S, New England Biolabs) was put into a round-bottom tube coated with the LGR5 protein, a gastric cancer cell marker. Then, only the attached phages were isolated, followed by repeatedly performing the same process three times, to select only phages having a high attachment ability. Among these, 48 phages were randomly selected to treat 96-well plates coated with LGR5 and BSA with each of the randomly selected phages. The resulting products were reacted with anti-M13-HRP, TMB, and stop solution (H2S04), and then the absorbance at a wavelength of 450 nm was measured.

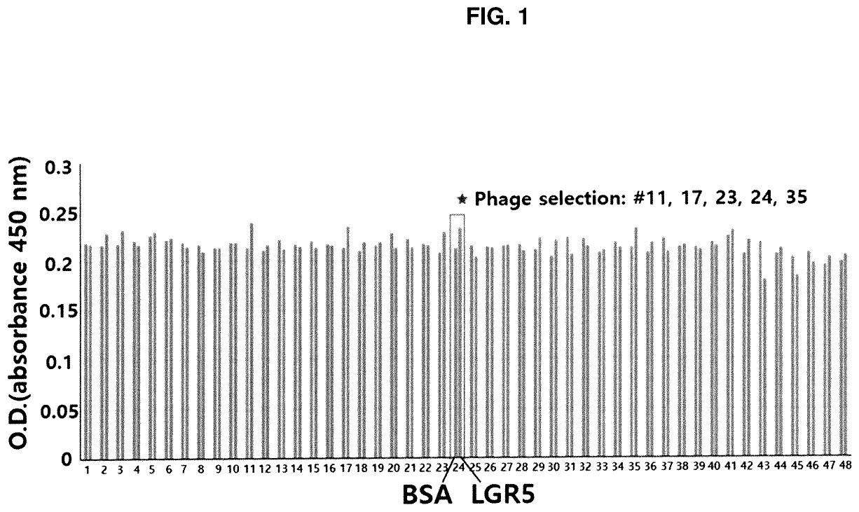

As a result, as shown in FIG. 1, only phages having absorbance optical density (O.D.) values higher in LGR5 than in BSA were selected. In addition, as a result of sequencing a peptide binding to phage No. 24, having the highest O.D. value among phage Nos. 11, 17, 23, 24, and 35, selected thereby, a 7-mer peptide sequence “STCTRSR” (SEQ ID NO: 1) being highly hydrophilic was confirmed.

### 2. Confirmation of Attachment Ability of Phage and Peptide to Gastric Cancer Cell Line

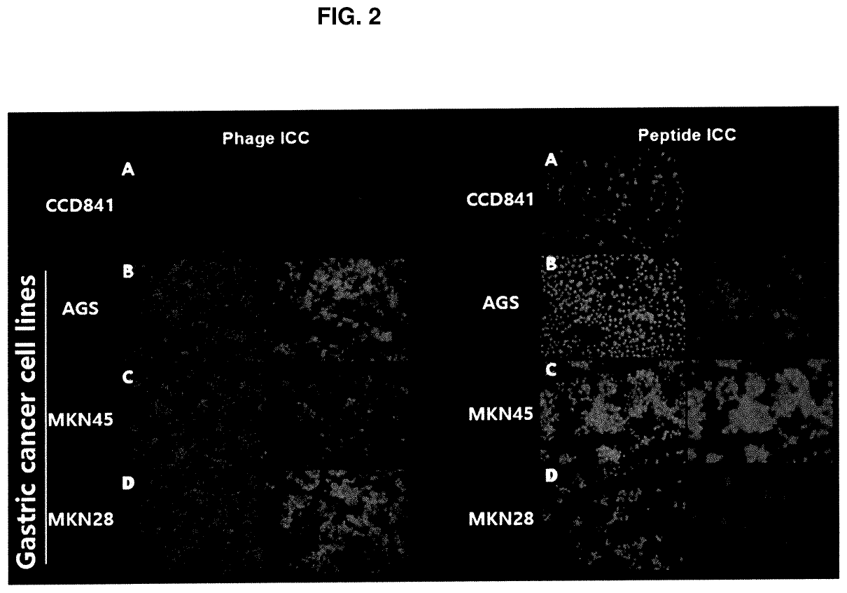

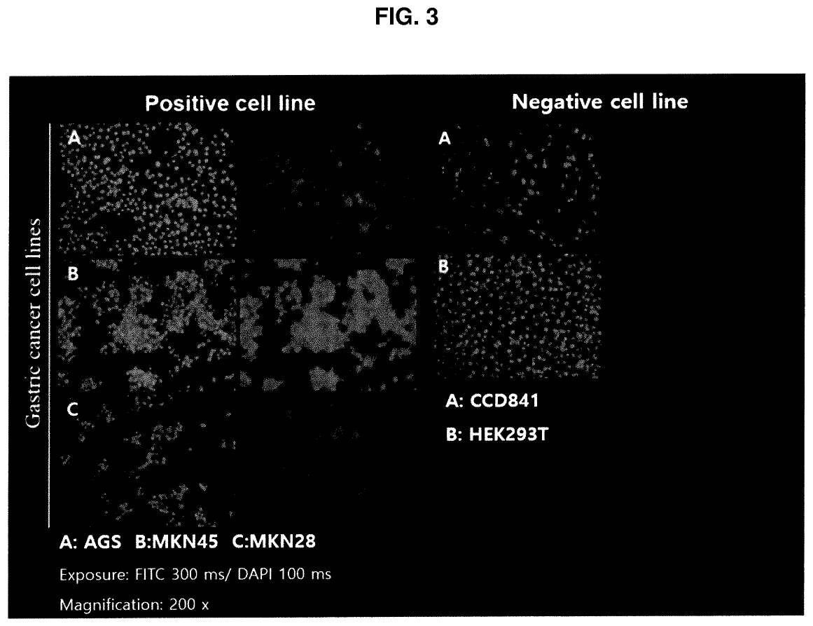

To confirm the attachment abilities, phage No. 24 and the peptide were each independently bound to FITC to perform immunochemical staining. As a result, as shown in FIG. 2, it was confirmed that the attachment ability to the phage and the peptide was better in the case of AGS, MKN45, and MKN28, the gastric cancer cell lines, than in the case of CCD841, the normal colonic epithelial cell line. In particular, the attachment ability was confirmed to be significantly good in the case of the MKN45 cell line. However, as shown in FIG. 3, the attachment ability was not able to be confirmed in the case of the HEK293T kidney cells not containing the LGR5 receptor.

### 3. Confirmation of Fluorescence Density Saturation of FITC-Peptide

To confirm the maximum fluorescence absorption capacity of FITC-peptide, CCD841 cells and MKN45 cells were attached to each 8-well chamber. Then, the chamber was treated with the peptide at concentrations of 1, 10, 25, 50, 100, 200, 400, and 800 uM.

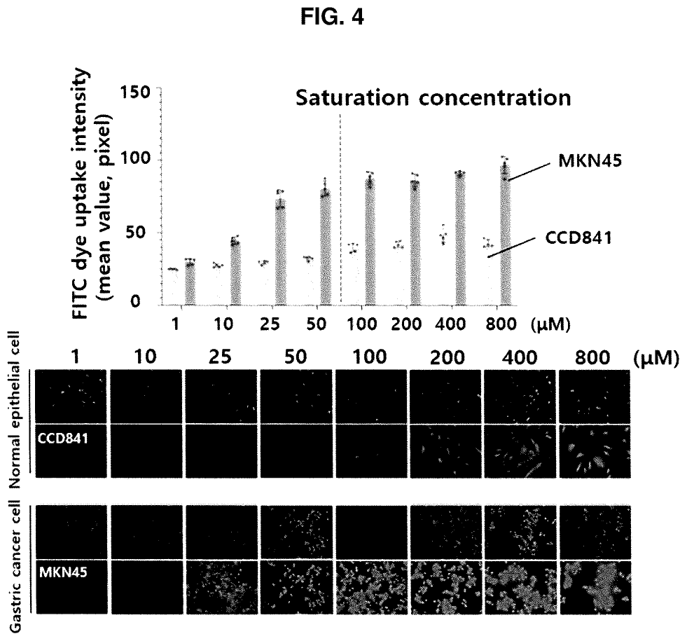

As a result, as shown in FIG. 4, it was confirmed that the difference in fluorescence density values based on the attachment ability of the peptide to the CCD841 cells and the MKN45 cells was the largest at a concentration of 25 UM, and the fluorescence density was saturated in all cells from a concentration of 100 UM or higher.

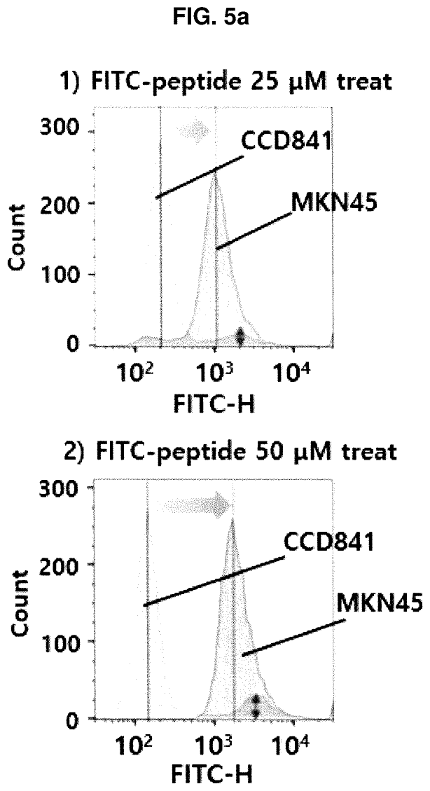

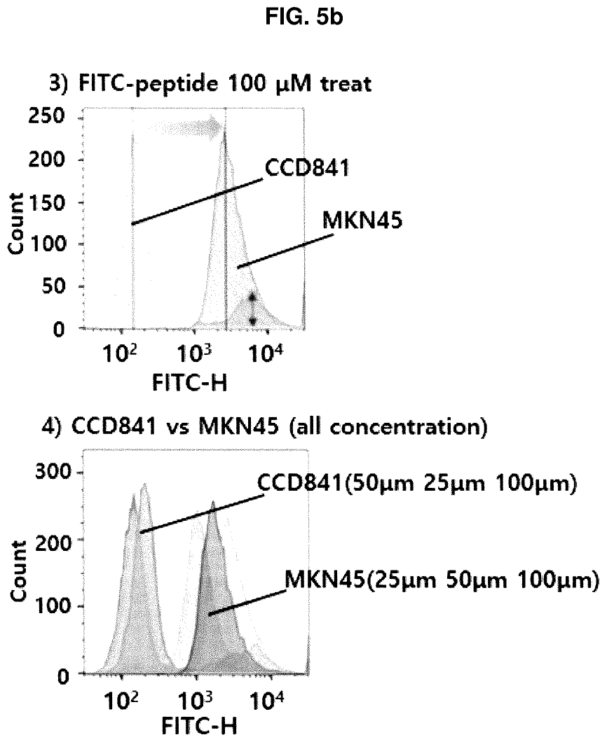

In addition, after treating each of the CCD841 cells and the MKN45 cells with FITC-peptide at a concentration of 25, 50, and 100 UM for 1 hour, the FITC fluorescence density was measured using a flow cytometer. As a result, as shown in FIGS. 5A and 5B, it was confirmed that the fluorescence density value increased in a concentration-dependent manner in the case of MKN45, the gastric cancer cells, compared to that in the case of CCD841, the normal cells.

### 4. Confirmation of Targeting Ability In Vivo of Peptide

Through Examples 1 to 3, the gastric cancer-targeting ability of the peptide was confirmed at the cellular level. Accordingly, to confirm the targeting ability in vivo, a xenograft model was created using MKN45 in 5-week-old immunodeficient mice. Then, Cy5.5-binding peptide was injected at a concentration of 50 nM into the tail vein.

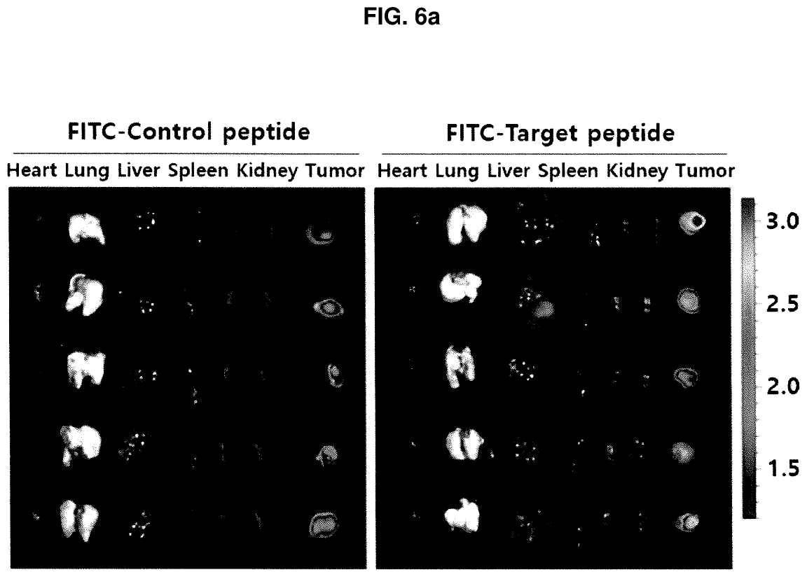

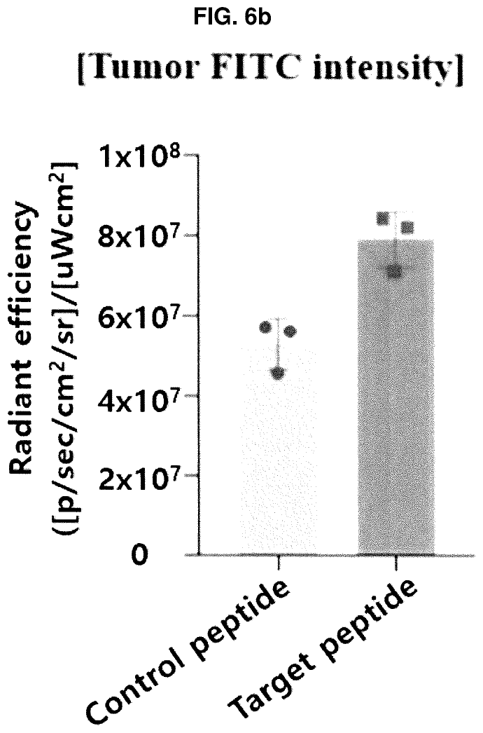

As a result, as shown in FIGS. 6A and 6B, although fluorescence was poorly detected in other organs such as the heart, lung, liver, spleen, and kidney, significantly high fluorescence density was detected in the tumor tissue. In addition, compared to the case of the control peptide group, a further better tumor-targeting ability was exhibited in the case of the developed target peptide group.

### 5. Confirmation of Photodynamic Therapy Efficacy of Peptide

### (1) Confirmation of Cellular Uptake of Chlorin E6

To confirm the tumor-targeting and therapeutic efficacies of the peptide, photodynamic therapy using chlorin e6 (Ce6), a photosensitizer, was performed. First, to confirm the degree of cellular uptake of Ce6-peptide, AGS, MKN45, and MKN28, gastric cancer cell lines, and CCD841 cells, normal cells, were each independently treated with the Ce6-peptide at a concentration of 10 uM.

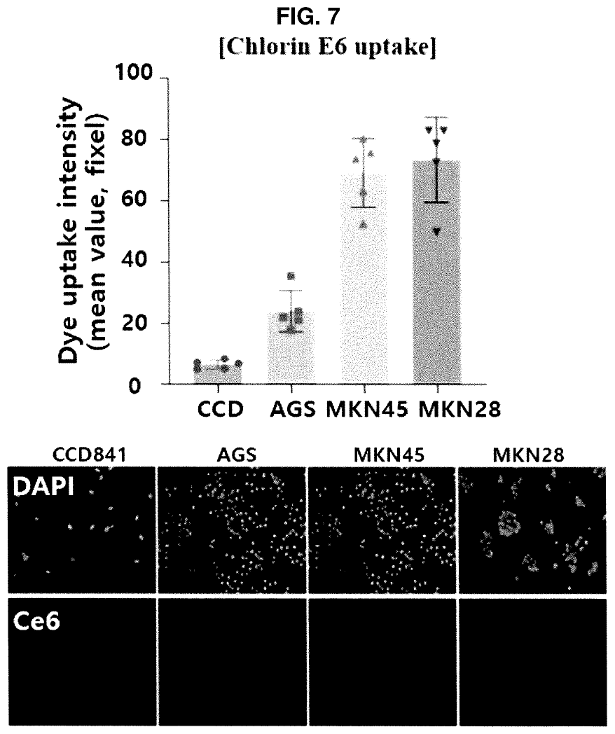

As a result, as shown in FIG. 7, the fluorescence density was measured to be higher in the case of the gastric cancer cell lines than in the case of the normal cell line, showing a difference in uptake of up to 11 times or more.

### (2) Cytotoxicity Analysis Through Photodynamic Therapy

To analyze cytotoxicity through photodynamic therapy, MKN45 cells were treated with Ce6 and the Ce6-peptide at concentrations of 0, 1, 5, 10, 20, 50, 100 nM, and 1 μM and then irradiated with a laser beam based on 10 J, 500 mW, and 3 cm for 2 minutes and 35 seconds.

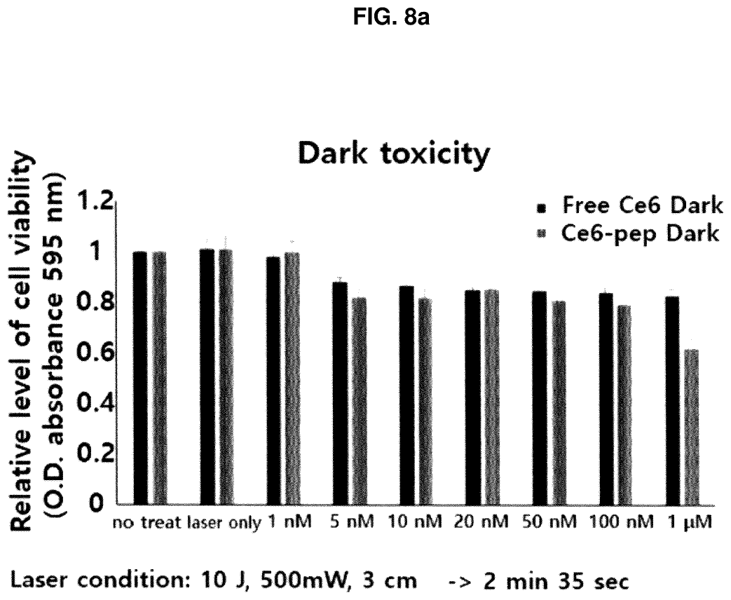

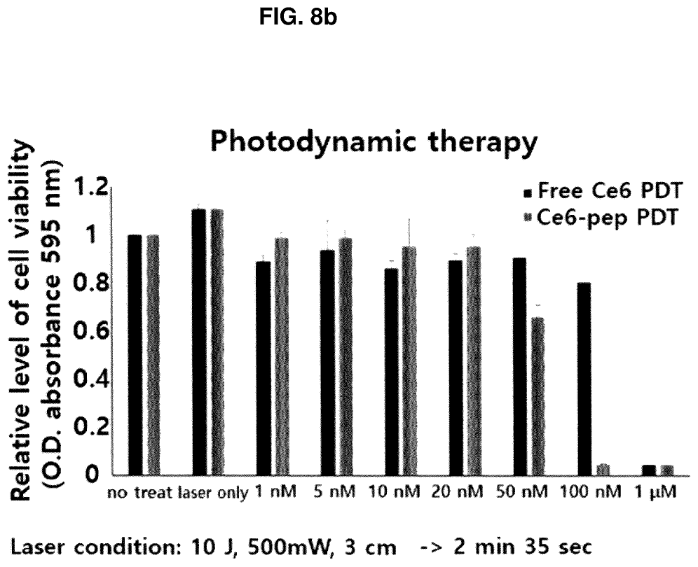

As a result, as shown in FIGS. 8A and 8B, in a dark toxicity experiment without involving laser irradiation, significant toxicity was not induced in either the group treated with Ce6 or the group treated with the Ce6-peptide. However, when treated at a concentration of 1 μM, 60% of the entire cells were killed in the case of the group treated with the Ce6-peptide.

In a photodynamic therapy experiment involving laser irradiation, cells were rapidly killed from a concentration of 50 nM or higher in the case of the group treated with the Ce6-peptide. However, cells were not killed in the case of the group treated with Ce6 alone. This confirmed that cell death was caused by the attachment ability of the peptide specific to cancer cells.

### (3) Confirmation of Whether Cell Death is Caused by ROS

To confirm whether cells were killed by photodynamic therapy, DCF-DA staining was performed to check whether cell death, the mechanism of the corresponding treatment, was caused by reactive oxygen species (ROS).

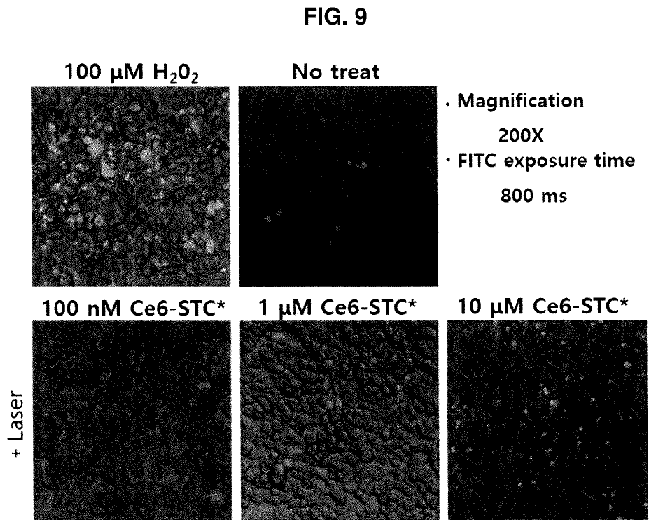

As a result, as shown in FIG. 9, it was confirmed that ROS was detected in the case of a positive control group, MKN45 cells treated with hydrogen peroxide, and was not detected in the case without involving any treatment. In addition, it was confirmed that ROS was detected in a concentration-dependent manner in the group treated with the Ce6-peptide and irradiated with a laser beam. This means that cancer cells were effectively killed by photodynamic action with laser irradiation performed on the photosensitizer-peptide composite.

### 6. Confirmation of Tumor-Targeting Ability of Ce6-Peptide

To confirm the tumor-targeting ability of Ce6-peptide, an in vivo targeting experiment was performed on an MKN45 xenograft mouse model. Ce6 and the Ce6-peptide were injected at a concentration of 5 mg/kg into the tail vein of mice in which a tumor with a diameter of about 8 mm was developed. After 4 hours, the concentration of Ce6 in the tumor was confirmed using an in vivo imaging system (IVIS).

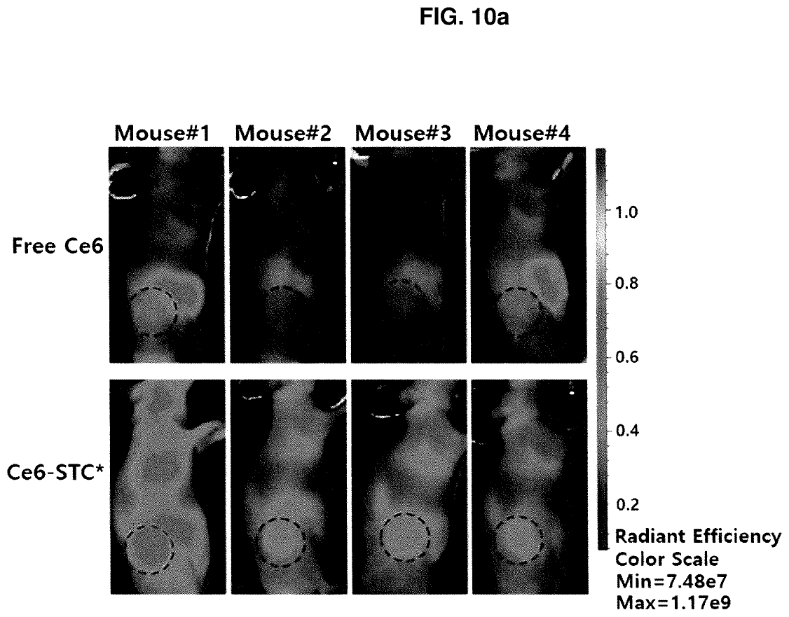

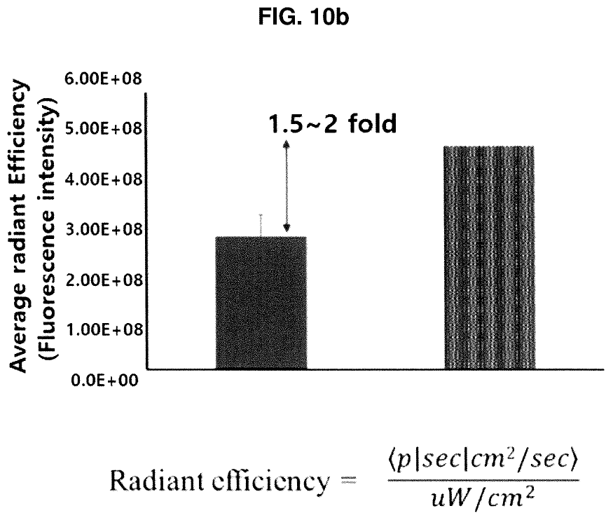

As a result, as shown in FIGS. 10A and 10B, the fluorescence intensity was confirmed to be 1.5 to 2 times higher in the case of the group treated with the Ce6-peptide than in the group treated with Ce6 alone.

In addition, organs were extracted from each subject 4 hours after the injection to analyze tumor-targeting ability ex vivo using an IVIS.

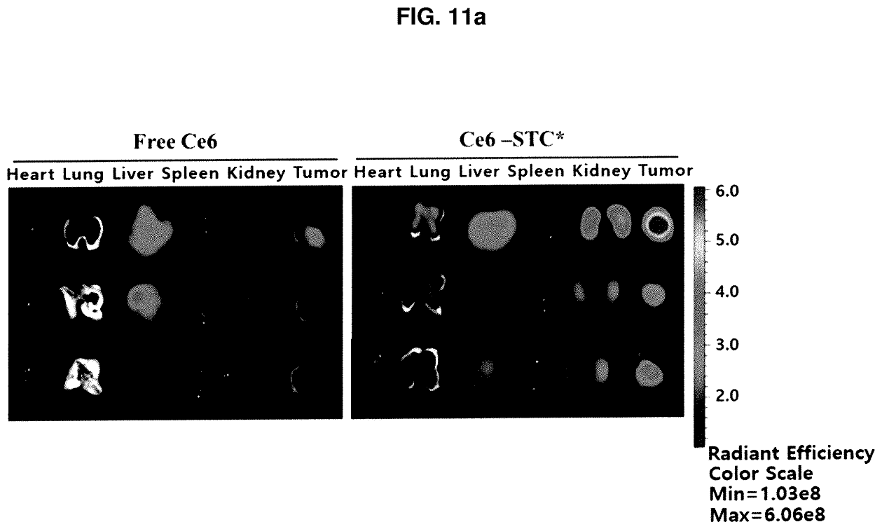

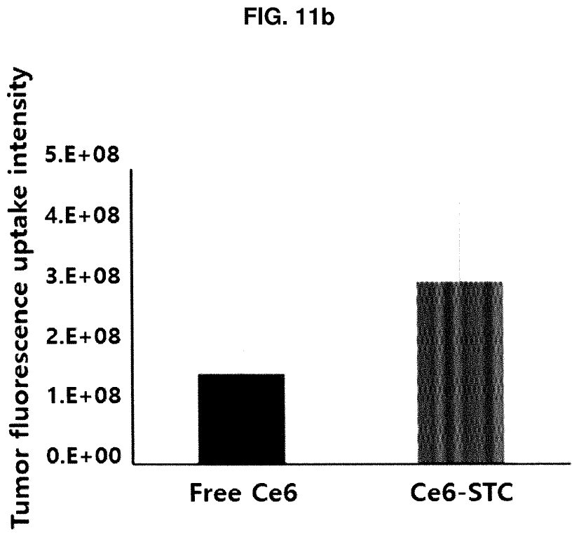

As a result, as shown in FIGS. 11A and 11B, it was confirmed that Ce6 was more infiltrated into the tumor than into other organs such as the heart, lung, liver, spleen, and kidney.

In particular, the tumor-targeting ability was significantly better in the case of the group treated with the Ce6-peptide than in the group treated with Ce6 alone.

Through the above results, it is seen that the tumor-targeting ability of the peptide further improves the tumor-infiltrating ability of the Ce-peptide composite, which suggests the possibility of not only tumor diagnosis but also treatment using a specific sequence.

### 7. Confirmation of Local Tumor Treatment Efficacy Through Peptide and Laser Irradiation

To confirm local tumor treatment efficacy using the peptide, an experiment was performed by dividing the mice in Example 6 into four groups: an untreated group, a group irradiated with a laser beam, a group irradiated with a laser beam after Ce6 administration, and a group irradiated with a laser beam after Ce6-peptide administration. Ce6 and the Ce6-peptide were administered at a concentration of 5 mg/kg into the tail vein. When 4 hours elapsed, the tumor area was irradiated with a laser beam for 9 minutes under conditions based on 200 J and 300 mW. Then, tumor size, body weight, tumor necrosis, and the like were observed at intervals of about 3 days. No change in body weight was observed during the treatment period.

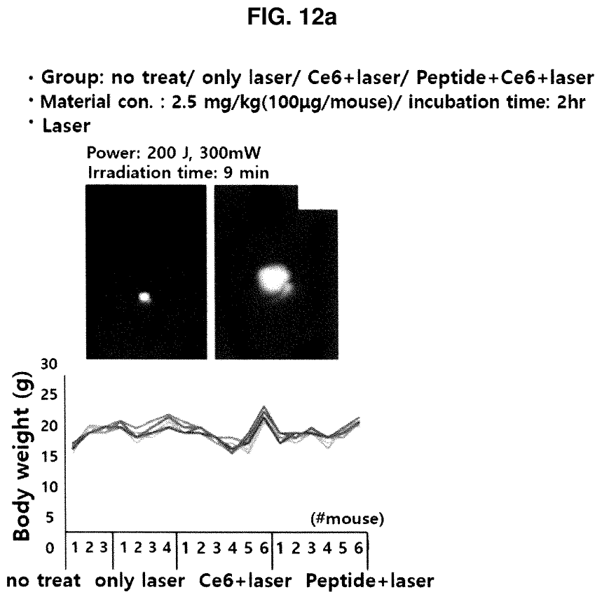

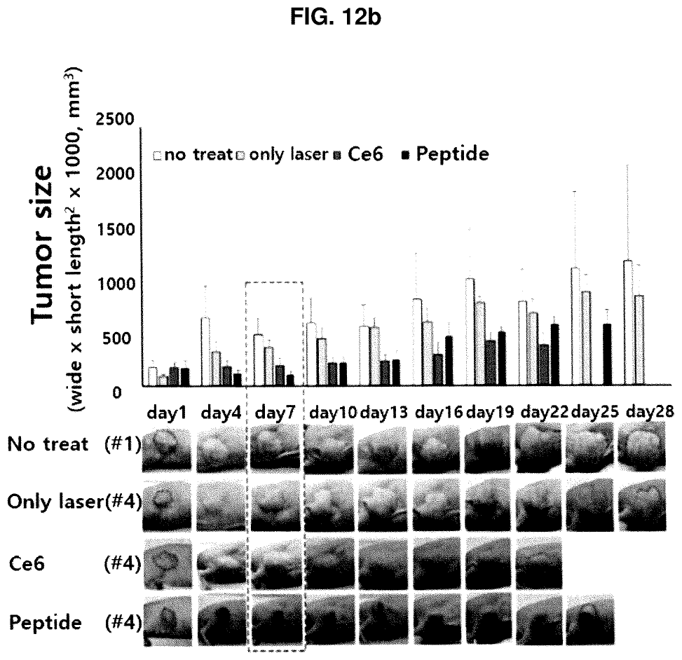

As a result, as shown in FIGS. 12A and 12B, in the case of the group treated with the Ce6-peptide, tumor necrosis was observed 2 days after the treatment. In addition, in the case of the group treated with the Ce6-peptide, the tumor size was confirmed to be significantly reduced on the 7th day compared to that in the case of the other groups. This confirmed that the local tumor was treatable through tumor targeting using the peptide and photodynamic response with the photosensitizer and laser irradiation.

### 8. Comparison of Tumor-Targeting Ability of Target Peptide and Negative Control Peptide

To confirm the tumor-targeting ability of the target peptide, the tumor tissue was treated with both the target peptide and a negative control peptide, not having the attachment ability to tumors, binding to FITC. The negative control peptide is a peptide made by randomly mixing the amino acid positions of the non-specific binding peptide sequence “QLMRPPV” (SEQ ID NO: 2).

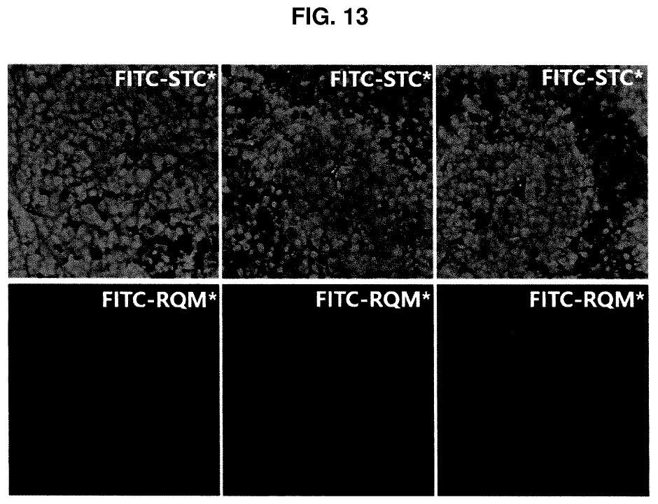

As a result, as shown in FIG. 13, when treated with the negative control peptide, the fluorescence based on FITC attachment was not confirmed. However, when treated with the target peptide, the fluorescence intensity was confirmed to be significantly high.

### 9. Verification of Peptide Targeting Ability in Intraperitoneal Metastasis Model

Through Example 6, the targeting ability in vivo of the peptide was confirmed in the xenograft model using MKN45, the gastric cancer cell line. Accordingly, to further verify the targeting ability in an intraperitoneal metastasis model, the same MKN45 cell line was injected intraperitoneally, and the targeting ability of the peptide was further verified.

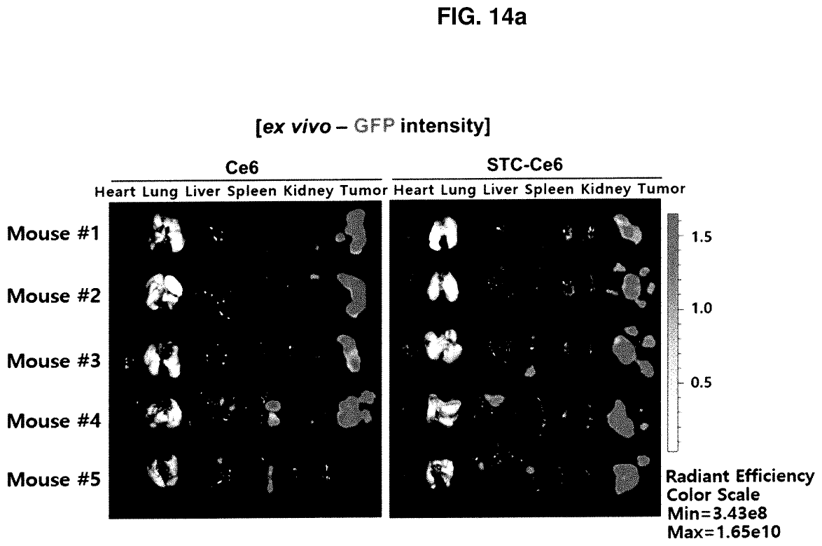

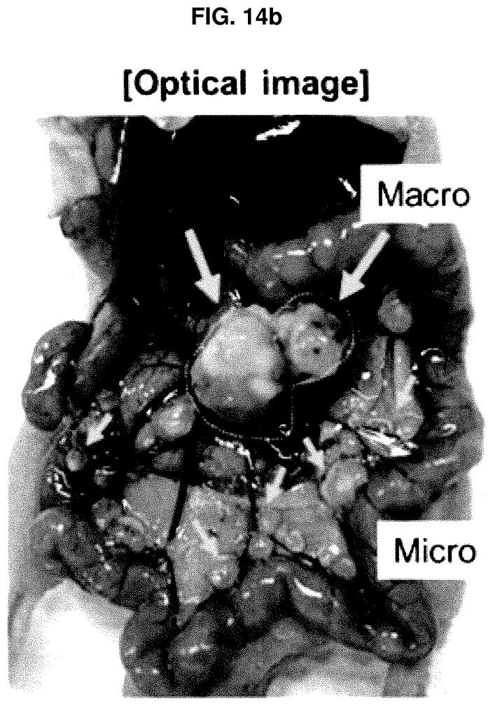

Intraperitoneal metastasis was confirmed within two weeks by injecting 1×10⁷ of the GFP-tagged MKN45 cell line into 5-week-old Balb/c nude mice. As a result, as shown in FIGS. 14A and 14B, additional infiltration of the cells into the liver, kidney, and spleen tissues and large tumors with a size of about 2 cm were observed.

To confirm the targeting ability of the peptide, modeled mice with intraperitoneal metastasis were treated with Ce6 and the Ce6-peptide at a concentration of 5 mg/kg and autopsied 4 hours after the treatment to confirm the concentration of the substance in the tissues using an IVIS.

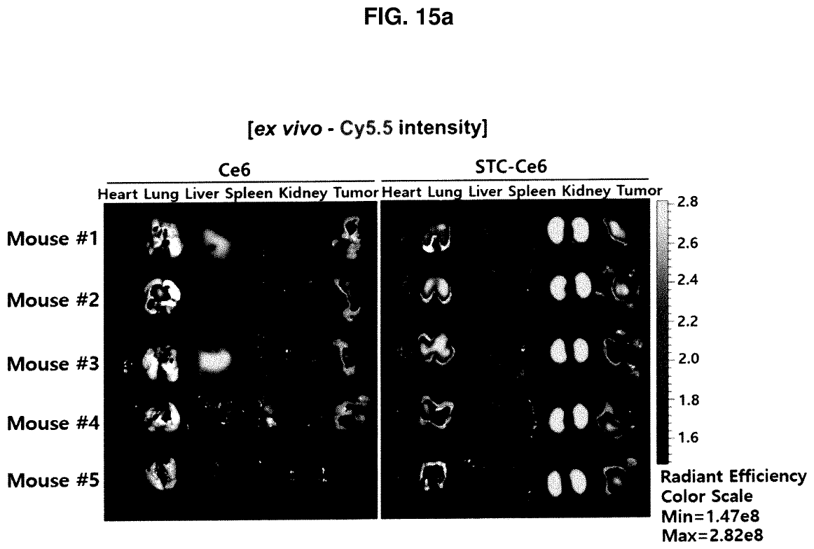

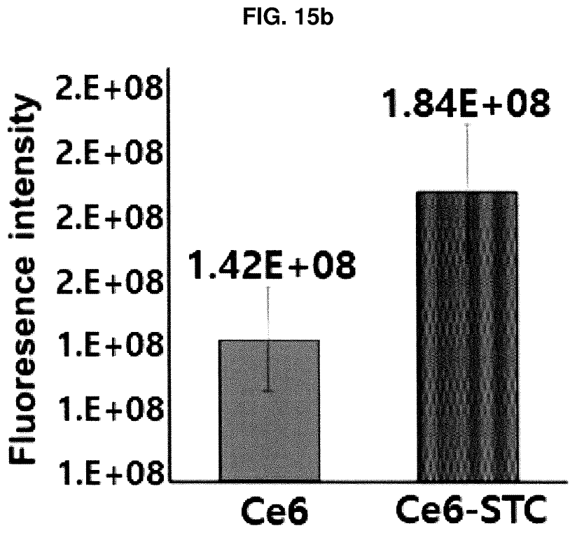

As a result, as shown in FIGS. 15A and 15B, it was confirmed that the tumor-targeting ability was better in the case of the group treated with the Ce6-peptide than in the case of the group treated with Ce6 alone. It was confirmed that due to the high concentration in the kidney, the Ce6-peptide was less likely to non-specifically bind to other organs and was more likely to be excreted from the body due to rapid circulation in the body, compared to Ce6 alone. These results confirmed that the peptide had the targeting ability not only in the xenograft model but also in the intraperitoneal metastasis models. This suggests that not only local tumors but also metastatic tumors are diagnosable.

### 10. Isotope Targeting of Intraperitoneal Metastatic Tumors

Although gastric cancer is known as a tumor that easily metastasizes to the abdominal cavity, a sensitive diagnosis of abdominal metastasis of gastric cancer has yet to be resolved. To resolve such a clinical dilemma, gastric cancer targeting was additionally researched using a composite of an isotope and a gastric cancer-specific peptide.

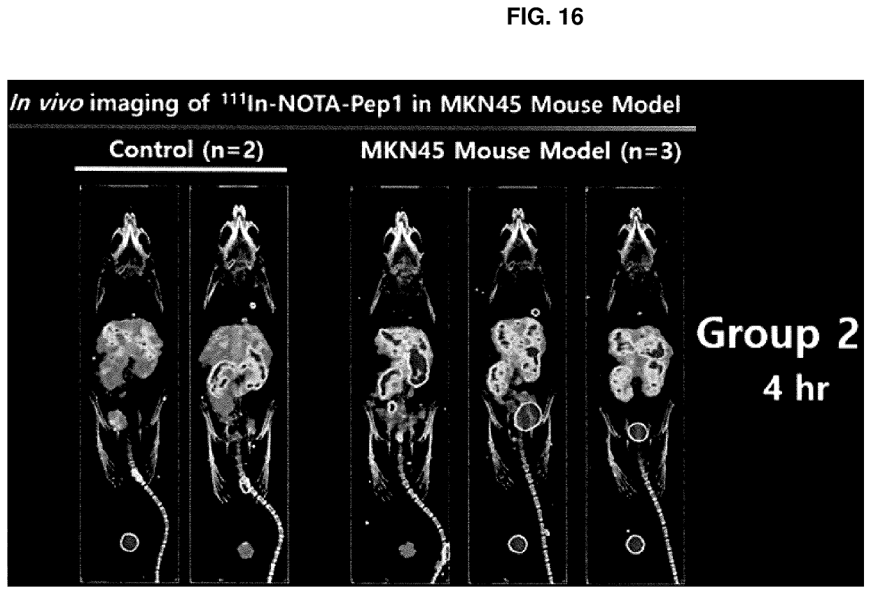

Due to the hydrophilic nature of the peptide, it was confirmed that when injected into a gastric cancer animal model, the isotope ¹¹¹In-peptide composite was mostly excreted through the kidneys and bladder. As shown in FIG. 16, higher fluorescence was observed in the abdominal cavity in the case of the MKN45 peritoneal metastasis model than that in the case of normal animals. This suggests that isotopic targeting of metastatic tumors in the abdominal cavity is enabled.

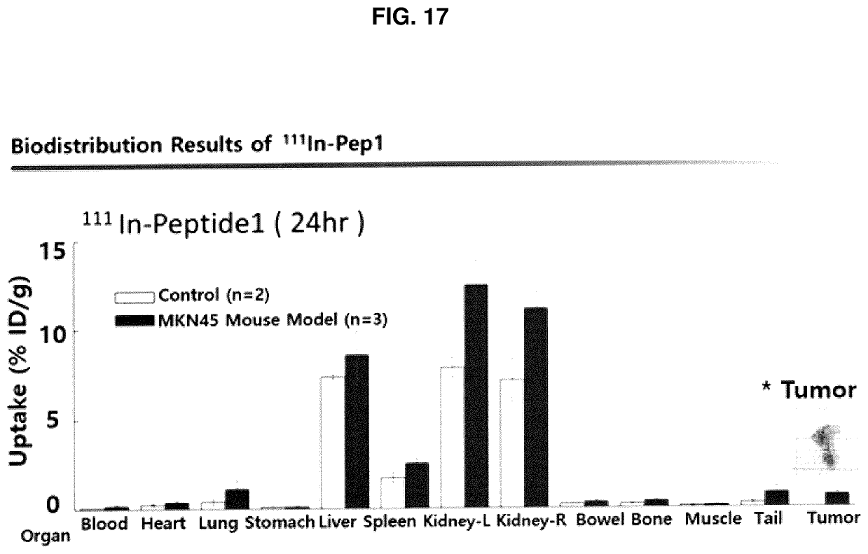

As shown in FIG. 17, the isotope-peptide distribution in tissues confirmed 24 hours after the injection was observed to be high in the liver and kidneys but insignificantly high in the tumor. However, this is believed to be because the hydrophilic nature of the peptide allows the isotope-peptide to be rapidly released.

1.-23. (canceled)

24. A method of diagnosing a cancer, the method comprising:
administering a composition comprising a polypeptide having an amino acid sequence of SEQ ID NO: 1 to a subject; and
confirming a position of the polypeptide in the subject.

25. The method of claim 24, wherein the composition further comprises a fluorescent material.

26. The method of claim 25, wherein the fluorescent material binds to the polypeptide.

27. The method of claim 25, wherein the fluorescent material comprises one or more selected from the group consisting of a xanthene derivative, a cyanine derivative, an oxadiazole derivative, an acridine derivative, an arylmethine derivative, a tetrapyrrole derivative, a near-infrared fluorophore (NIR fluorophore), chlorin e6 (Ce6), and green fluorescent protein (GFP).

28. The method of claim 27, wherein the xanthine derivative comprises one or more selected from the group consisting of fluorescein, Oregon Green, and Texas Red;
the cyanine derivative comprises one or more selected from the group consisting of cyanine 2 (Cy2), Cy3, Cy3B, Cy3.5, Cy5, Cy5.5, Cy7, indocarbocyanine, rhodamine, oxacarbocyanine, thiacarbocyanine, and merocyanine;
the oxadiazole derivative comprises one or more selected from the group consisting of pyrodyloxazole, nitrobenzoxadiazole, and benzoxadiazole;
the acridine derivative comprises one or more selected from the group consisting of Nile red, Nile orange, and acridine yellow;
the arylmethine derivative comprises one or more selected from the group consisting of aumarine, crystal violet, and malachite green;
the tetrapyrrole derivative comprises one or more selected from the group consisting of porphin, phthalocyanine, and bilirubin; and
the NIR fluorophore comprises one or more selected from the group consisting of X-SIGHT, Pz 247, DyLight 750, DyLight 800, Alexa Fluor 680, Alexa Fluor 750, IRDye 680, IRDye 800CW, indocyanine green, and a zwitterionic near-infrared fluorophore.

29. The method of claim 24, wherein the cancer expresses leucine-rich repeat-containing G-protein coupled receptor 5 (LGR5) protein.

30. The method of claim 24, wherein the polypeptide binds to LGR5 protein of the cancer.

31. The method of claim 24, wherein the cancer comprises one or more selected from the group consisting of gastric cancer, colon cancer, pancreatic cancer, liver cancer, cervical cancer, breast cancer, ovarian cancer, head and neck cancer, carcinoid, prostate cancer, lung cancer, bladder cancer, endometrial cancer, melanoma, kidney cancer, testicular cancer, glioma, thyroid cancer, skin cancer, and lymphoma.

32. The method of claim 24, wherein the cancer is a metastatic tumor.

33. The method of claim 32, wherein the polypeptide binds to an isotope.

34. The method of claim 33, wherein the isotope comprises one or more selected from the group consisting of ¹¹C, ¹³N, ¹⁸F, ⁶⁸Ga, ⁶¹Cu, ¹²⁴I, ¹²⁵I, ¹¹¹In, ^{99m}Tc, ³²P, and ³⁵S.

35. A method of preventing or treating a cancer, the method comprising:
administering a composition comprising a polypeptide having an amino acid sequence of SEQ ID NO: 1 to a subject in need thereof; and
irradiating the subject with light.

36. The method of claim 35, wherein the composition further comprises a fluorescent material.

37. The method of claim 36, wherein the fluorescent material binds to the polypeptide.

38. The method of claim 36, wherein the fluorescent material comprises one or more selected from the group consisting of a xanthene derivative, a cyanine derivative, an oxadiazole derivative, an acridine derivative, an arylmethine derivative, a tetrapyrrole derivative, a near-infrared fluorophore (NIR fluorophore), chlorin e6 (Ce6), and green fluorescent protein (GFP).

39. The method of claim 38, wherein the xanthine derivative comprises one or more selected from the group consisting of fluorescein, Oregon Green, and Texas Red;
the cyanine derivative comprises one or more selected from the group consisting of cyanine 2 (Cy2), Cy3, Cy3B, Cy3.5, Cy5, Cy5.5, Cy7, indocarbocyanine, rhodamine, oxacarbocyanine, thiacarbocyanine, and merocyanine;
the oxadiazole derivative comprises one or more selected from the group consisting of pyrodyloxazole, nitrobenzoxadiazole, and benzoxadiazole;
the acridine derivative comprises one or more selected from the group consisting of Nile red, Nile orange, and acridine yellow;
the arylmethine derivative comprises one or more selected from the group consisting of aumarine, crystal violet, and malachite green;
the tetrapyrrole derivative comprises one or more selected from the group consisting of porphin, phthalocyanine, and bilirubin; and
the NIR fluorophore comprises one or more selected from the group consisting of X-SIGHT, Pz 247, DyLight 750, DyLight 800, Alexa Fluor 680, Alexa Fluor 750, IRDye 680, IRDye 800CW, indocyanine green, and a zwitterionic near-infrared fluorophore.

40. The method of claim 34, wherein the cancer expresses leucine-rich repeat-containing G-protein coupled receptor 5 (LGR5) protein.

41. The method of claim 34, wherein the polypeptide binds to LGR5 protein of the cancer.

42. The method of claim 34, wherein the cancer comprises one or more selected from the group consisting of gastric cancer, colon cancer, pancreatic cancer, liver cancer, cervical cancer, breast cancer, ovarian cancer, head and neck cancer, carcinoid, prostate cancer, lung cancer, bladder cancer, endometrial cancer, melanoma, kidney cancer, testicular cancer, glioma, thyroid cancer, skin cancer, and lymphoma.

43. The method of claim 34, wherein the cancer is a metastatic tumor.

44. The method of claim 43, wherein the polypeptide binds to an isotope.

45. The method of claim 44, wherein the isotope comprises one or more selected from the group consisting of ¹¹C, ¹³N, ¹⁸F, ⁶⁸Ga, ⁶¹Cu, ¹²⁴I, ¹²⁵I, ¹¹¹In, ^{99m}Tc, ³²P, and ³⁵S.


SEQUENCE LISTING (2 sequences)

SEQ ID NO: 1 - protein, 7 aa, Artificial Sequence
  LGR5-targeting peptide
STCTRSR

SEQ ID NO: 2 - protein, 7 aa, Artificial Sequence
  negative control peptide
QLMRPPV